(19) United States
(12) Patent Application Publication
(10) Pub. No.: US 2015/0157728 A1
(43) Pub. Date: Jun. 11, 2015

(54) STABILIZED AND SOLUBILIZED DRUG FORMULATION FOR TOPICAL APPLICATION AND TRANSDERMAL EFFICACY FOR COSMETIC IMPROVEMENT AND METHODS OF FORMULATION

(71) Applicant: PANKAJ MODI, Ancaster (CA)

(72) Inventor: PANKAJ MODI, Ancaster (CA)

(21) Appl. No.: 14/101,983

(22) Filed: Dec. 10, 2013

(51) Int. Cl. A61K 47/42; A61K 31/728; A61Q 19/08; A61K 8/73; A61K 47/18; A61K 8/64; A61K 38/48; A61K 8/66

(52) U.S. Cl. CPC A61K 47/42; A61K 38/4893; A61K 31/728; A61K 8/66; A61K 8/735; A61K 47/183; A61K 8/64; A61Q 19/08

(57) ABSTRACT

The invention relates to a novel stabilized and solubilized topical formulation for cosmetic improvements and methods of making the same comprising multiplexed molecular penetration enhancers and essential and semi-essential amino acid protein binders for the topical application and transdermal delivery of one or more active ingredients and/or pharmaceutical agents. The invention further relates to the use of the topical formulation in connection with the providing of cosmetic improvements in individuals.

### PRIORITY CLAIM

THIS APPLICATION CLAIMS THE PRIORITY OF AND IS A CONTINUATION IN PART OF U.S. patent application Ser. No. 11/057,481 FILED Feb. 14, 2005, U.S. patent application Ser. No. 12/133,939 FILED Jun. 5, 2008, U.S. patent application Ser. No. 12/126,594 FILE Sep. 11, 2008 AND U.S. patent application Ser. No. 12/803,544 FILED Jun. 19, 2010, EACH OF WHICH IS INCORPORATED HEREIN IN THEIR ENTIRETY AND EXPRESSLY BY REFERENCE AS THOUGH SET FORTH IN FULL.

### FIELD OF THE INVENTION

The present invention relates to the field of drug formulations for use by means of topical application to provide a transdermal delivery system for a novel stabilized and solubilized topical formulation for cosmetic improvements and methods of making the same comprising multiplexed molecular penetration enhancers in conjunction with essential and semi-essential amino acid protein binders for topical application and transdermal delivery of one or more active ingredients and/or pharmaceutical agents. The invention further relates to the use of the topical formulation and methods for preparing and using these pharmaceutical compositions in connection with the providing of cosmetic improvements in humans, as well as methods for enhancing the stability and the rate of absorption of the therapeutic agent.

### BACKGROUND OF THE INVENTION

Wrinkles mostly result from a strong muscular contraction or from a prolonged time in a frowning or contracted position. The several available (marketed) topically applicable compounds which act on wrinkles are generally 5000 times weaker than Botox and are hardly toxic. However, their muscle-relaxing effect is too weak and too inconstant to allow a satisfying wrinkle-reducing effect. A further disadvantage is their insufficient proteolytic stability.

Today such mimic and age-related wrinkles are often treated with Botox (Botulinum toxin A). Botox is injected in the muscles which are thereby paralyzed. The muscles at the eyes or at the forehead do not operate any more, making the appearance of a forehead wrinkle impossible. However, the fact that the treatment with subcutaneously injected Botox has to be conducted by a doctor, its consequently high cost and its extremely high toxicity constitute considerable disadvantages of Botox. Its effectiveness lasts from 3 to 6 months, whereupon the treatment has to be repeated.

The mechanism of action of Botox consists in selectively blocking the acetylcholine release at the neuromuscular synapsis, leading to muscle paralysis. This occurs through splitting of a protein, the so-called SNAP-25. The N-terminal amino acid sequence of SNAP-25 (H-Glu-Glu-Met-Gln-Arg-Arg-NH2) also inhibits the Ca++-dependent neurotransmitter release in the synapses and leads to muscle relaxation (EP1 180 524).

Ironically, it is this “paralytic” property that has led to the development of therapeutic uses for botulinum toxin beginning in the 1960's. In fact, botulinum toxin is now safely used in the treatment of over a dozen human diseases involving hyperactive skeletal muscles. More generally, pharmaceutical preparations of botulinum toxin are used for the treatment of neurological disorders, muscle dystonias, smooth muscle disorders, autonomic nerve disorders, headaches, wrinkles, sports injuries, cerebral palsy, spasms, tremors and pain.

Botulinum toxin is an extremely potent neurotoxin produced by the bacteria Clostridium botulinum. The toxin acts as a chemodenervating agent by inhibiting the release of the neurotransmitter acetylcholine, thereby preventing synaptic transmission across the neuromuscular junction and inhibiting muscular contraction to cause temporary paralysis.

Historically, botulinum toxin has been used for the correction of neurological and neuromuscular disorders, such as hemifacial spasm, adult onset spasmodic torticollis, anal fissure, blepharospasm, cerebral palsy, cervical dystonia, migraine headaches, and strabismus. More recently, botulinum toxin has proven useful for certain dermatologic and cosmetic indications, such as the management of hyperhidrosis, facial rhytides (wrinkles), and other disorders resulting from spasms or from contractions of facial muscles.

While the use of botulinum toxin to treat wrinkles is currently one of the most popular cosmetic treatments, the conventional method of administering toxin for this purpose by injecting the toxin into a patient gives rise to several problems. First, botulinum toxin typically must be injected into multiple sites in order to treat a given wrinkle. The selection of the particular sites of injection is not easy and must be determined by a skilled practitioner with a deep understanding of muscle anatomy. The injections, which are performed along the muscle or muscles responsible for forming the wrinkle (rather than along the wrinkle itself), must be done with proper technique. Improper injection technique can lead to undesirable effects, including the unintended spread of the toxin away from the injection site and to adjacent muscles, thereby weakening the muscle and affecting facial expression or function. Eyelid ptsosis (drooping eyelid), for example, can result when improper injection technique is used when treating forehead lines.

Furthermore, the multiple injections required to treat a single wrinkle can be painful, and injections can result in bruising and/or irritation around the injection site. The pain or anticipated pain associated with the injections can lead to anxiousness, stress or embarrassment in patients, thereby affecting their quality of life. Moreover, an entire patient population that could potentially benefit from the use of such chemodenervating agents remains untreated due to severe needle-related phobias or aversions.

Moreover, the effects of most chemodenervating agents, such as botulinum toxin, are temporary. The effects of injected botulinum toxin typically last between three and six months, after which the paralyzed nerve recovers and re-innervates the muscle by forming new nerve branches. Therefore, as the paralysis subsides, a patient is faced with the prospect of undergoing additional painful injections. With the current available technology, a patient must receive periodic injections indefinitely in order to achieve and maintain the desired results.

Accordingly, there is a need for improved methods of administering potent chemodenervating agents, such as botulinum toxin, for treating wrinkles. Specifically, there is a need for an efficacious, less painful method of delivering chemodenervating agents such as botulinum toxin to a patient for reducing the appearance of wrinkles. However, in order to accomplish this, the drug must be able to penetrate the skin in sufficient quantity to be efficacious but not cause a toxic reaction.

Drug penetration is hampered by the relatively low permeability of skin because the barrier properties of the skin allow only for the passage of small, uncharged or polar molecules, such as diatomic oxygen, glycerol, or water. Accordingly, polar molecules larger than water and charged molecules, such as certain amino acids or hydrogen ions, generally do not diffuse across the skin. See Cooper, G. M., The Cell: A Molecular Approach. Chapter 2 “The Chemistry of Cells,” p. 81, ASM Press, Washington D.C. (1997). Thus, therapeutically relevant rates of drug delivery often are difficult to achieve by applying a drug to the surface of the body because typical drugs are too large and/or charged to readily diffuse through the skin.

Pegylated botulinum toxin (botulinum toxin covalently coupled to polyethylene glycol) has been developed for the treatment of neuromuscular disorders. Pegylation of the toxin is site directed, thereby reducing antigenicity without interfering with the neurotoxic effect. (See, U.S. Patent Publication No. 20020197278). Also, hybrid-toxin molecules with reduced antigenicity have been synthesized using the targeting and internalization portion (heavy chain) of one toxin serotype and the catalytic portion of a different serotype (light chain). The hybrid-toxin molecules exhibit reduced antigenicity but retain the inherent-binding specificity of the botulinal-heavy chain from the first serotype and the catalytic potency of the light chain from the second serotype. (See, U.S. Pat. No. 6,444,209).

Reduced antigenicity may also be achieved by further purifying the neurotoxin by reducing the antigenic complex proteins and other clostridial proteins associated with the toxin. (See, U.S. Pat. Nos. 5,756,468 and 5,512,547). Type A neurotoxin produced by C. botulinum is present as part of a complex of at least seven different non-covalently bound proteins. These nontoxic proteins range in size from about 17 to 118 kD and are associated with the neurotoxin that has a molecular weight of about 147 kD. (Goodnough et al. (1993) Appl. Environ. Microbiol. 59: 2339-2342; Gimenez et al. (1993) Protein Chem. 12: 349-361; DasGupta (1980) Canad. J. Microbiol. 26: 992-997).

Some of the non-toxic proteins associated with the various toxin complexes have hemagglutinating abilities (Sugiyama (1980) Microbiol. Rev. 44: 419-448; Somers et al. (1991) J. Protein Chem. 10:415-425). In particular, non-neurotoxic fractions of the L complexes of type A, B, C, and D have been shown to have hemagglutinating activity. Hemagglutinin fractions isolated from the different serotypes show some serological cross-reactivity. Non-toxic fractions from type A and B serotypes cross-react (Goodnough and Johnson (1993) Appl. Environ. Microbiol. 59: 2339-2342) as do non-toxic fractions from types E and F. The non-toxic fractions of types C1 and D are antigenically identical as determined by Ouchterlony diffusion (Sakaguchi et al. (1974) Jpn. J. Med. Sci. Biol. 27: 161-170). By removing these proteins, more neurotoxin may be delivered to a therapeutic site with less antigenic proteins that may lead to the production of neutralizing antibodies.

Botulinum toxin is most frequently administered as a therapeutic agent by injecting a composition containing botulinum toxin into a patient using a needle or syringe. However, other modes of administration have been considered for botulinum toxins as well as botulinum toxins coupled with non-botulinum toxin receptor legends. Some modes of administration include topical application of botulinum toxin (e.g., see U.S. Pat. Nos. 6,063,768; 5,670,484; and German Patent Publication DE 198 52 981). German Patent Publication DE 198 52 981 discloses a composition containing botulinum toxin type A and a 50% dimethyl sulphoxide (DMSO) solution for the treatment of hyperhydrosis. Although DE 198 52 981 discusses that botulinum toxin may be used to treat hyperhydrosis by being topically applied to the skin, it is unclear whether the botulinum toxin permeated through the epidermis of the person, or if the effects were mediated by botulinum toxin passing through pores of the sweat glands. In any case, although DE 198 52 981 discloses that topical administration of botulinum toxin in a DMSO solution can be used to treat hyperhydrosis, compositions containing DMSO are not desirable because DMSO can irritate the skin.

In addition, although U.S. Pat. No. 5,670,484 discloses topical application of botulinum toxin to treat skin lesions, it does not disclose a composition containing botulinum toxin and an enhancing agent, as described herein. Furthermore, U.S. Pat. No. 5,670,484 only discloses that topical administration of botulinum toxin may inhibit cell proliferation. It is silent to topical application of botulinum toxin to treat disorders associated with neurosecretion of intracellular molecules. See also WO 00/15245 and Grusser Von O-J., Die ersten systematischen Beschreibungen and tierexperimentellen Untersuchungen des Botulismus, Sudhoffa Archiv (1986), 70(2), 167-186.

### SUMMARY OF THE INVENTION

It has been discovered that compositions comprised of botulinum toxin, as well as other toxins which have chemodenervating properties, and tripeptides, tripeptide-like compounds and derivatives thereof (hereinafter jointly referred to as “compounds of the present invention”) form low viscosity, topically applicable compositions that may be use for the treatment of mimic and age-related wrinkles to reach their site of action, the neuromuscular synapsis, rapidly and in sufficient concentration, block the synapsis and thereby induce a muscle-relaxing effect. The compounds (tri, tetra and penta and hexa peptides combinations) of the present invention have been discovered to permit and create a clearly better activity profile with regard to muscle relaxation and a higher proteolytic stability than Botulinum Toxin A alone and to help stabilized the Toxin when combined with this mixture of peptides. By way of example, Acetyl Hexapeptide-3 is one of the multiplexed peptides which may be employed to accomplish the objects of the invention.

It is a further object of the invention to go beyond merely limiting the neurotransmitters that tells facial muscles to move (a process known as exocytosis), but to do so in conjunction with a composition which minimizes potential complications, such as systemic toxicity or botulism poisoning, even upon administration of relatively high dosages, since the stratum corneum of the skin still retains some impermeability. Thus, it is an object of the invention to provide dosages of botulinum toxin (including types A, B, C, D, E, F, or G) that can range from as low as about 1 unit to as high as about 20,000 units, without fear of adverse side effects that may threaten the patient. The particular dosages may vary depending on the condition being treated, and the particular enhancing agent and therapeutic regime being utilized. For example, treatment of subdermal, hyperactive muscles may require high dosages (e.g., 1000 units to 20,000 units) of botulinum toxin topically applied in a composition containing an enhancing agent.

In comparison, treatment of neurogenic inflammation or hyperactive sweat glands may require relatively small topical dosages (e.g. about 1 unit to about 1,000 units) of botulinum toxin. Most preferably, the botulinum toxin is present in an amount so that between about 0.1 unit and about 5 units pass through the patient's skin to a subdermal target.

It is a further object of the invention to permit the therapeutic effects of the toxin in the composition to persist by permitting the slow release of the toxin by transdermal passage after topical application. Thus, the effects of a topical application which does not penetrate the skin with slow release toxin can persist for between about 2 months to about 6 months when administration is of a low viscosity or aqueous solution of the neurotoxin. However, it is a further object of the invention to permit the efficacious nature of the toxin to be present for up to about five years when the neurotoxin is administered topically in a composition that retains the toxin and slowly releases the toxin after it has passed through the skin.

It is a still further object of the present invention to provide muscle-relaxing compounds to be applied as topical actives against mimic and age-related wrinkles, the action of which is based on the inhibition of the acetylcholine receptor and which does present the disadvantages of Botox and of Botulinum A. The compounds of the present invention provide chemodernervation such that the nerves that send signals to facial muscles are inhibited thereby limiting subtle facial expressions and concomitantly reducing wrinkles. Moreover, the muscles will also be relieved of lingering tension, and the skin will relax as well. In short, the peptides are a mimic of the N-terminal end of SNAP-25 which competes with SNAP-25 for a position in the SNARE complex, thereby modulating its formation and serving as muscle relaxants. If the SNARE complex is slightly destabilized, the vesicle cannot release neurotransmitters efficiently and therefore muscle contraction is attenuated, preventing the formation of lines and wrinkles. In general the peptides of the composition, once delivered to the SNARE complex, help inhibit neurotransmitter signals from specific receptors thus reducing muscle contractions and inhibiting the wrinkles formation.

It is a further object of the invention to employ essential and semi-essential amino acids to stabilize the toxin and permit longer term release of the chemodenervating effects of the toxin to be achieved transdermally.

It is a further object of the invention to use chemical agents with the compositions of the invention to enhance penetration through the skin. Such chemical agents may include surfactants, lipids and other aliphatic compounds, liposomes and niosomes. While these compounds increase drug absorption through the skin to some extent, problems with developing pharmaceutically acceptable, stable formulations of both the delivery vehicle and the botulinum toxin harbored within can occur.

It is a further object of the invention to help avoid these problems by employing micro-emulsion formulations of topical agents to increase the absorption coefficient over those of conventional “oil and water” emulsion-based creams. Such micro-emulsion formulations may be employed to increase drug delivery of the botulinum toxin for patients who present exceptional indications.

It is a further object of the invention to employ compounds such as hyaluranidase to assist drug delivery and accelerate the absorption of topical botulinum.

### DETAILED DESCRIPTION OF THE INVENTION

The present invention is directed to stable low viscosity or liquid formulations of chemodenervating agents, such as botulinum toxin and other appropriate toxins, for use in pharmaceutical preparations. The formulations of the present invention have the advantage that, unlike currently available formulations, they are stable in low viscosity or liquid form during storage for protracted periods in excess of one year at standard refrigerator temperatures (approximately 4±2° C., or about 2-8° C., or, more generally, ranging from about 0-10° C.) and are also stable in low viscosity or liquid form during storage at “room temperature” which is about 25°, or more generally in the range of 10-30° for a period in excess of six months. Such formulations are particularly useful in conditions in which reduction or inhibition of cholinergic nerve input to a region, particularly a muscle or muscle group, gland or organ is ameliorative.

The term “wrinkle” refers to a fold or crease in the skin. Wrinkles can vary in size and intensity, from fine lines to deep furrows. Fine wrinkles encompass “crinkles” as well as lines which have a shallow trough and typically do not have significant ridging; ridging refers to the raising of the wrinkle edge above the adjacent plane of unwrinkled skin; fine wrinkles typically do not cast a shadow from light illuminated at an angle across the wrinkle. Deep wrinkles tend to have both a trough which is below the plane of the adjacent skin as well as ridging which extends above the plane of the adjacent unwrinkled skin. Deep wrinkles typically can cast shadows when illuminated with an appropriate light source at an angle across the wrinkle. Wrinkles in skin may be classified into three different types: dynamic wrinkles, static wrinkles and wrinkle folds. Dynamic wrinkles are caused by repeated contractions of muscles underlying the skin. For example, frowning or furrowing causes wrinkles between the eyebrows (glabellar lines or “frown lines”), raising of the eyebrows causes the horizontal lines alone the forehead (forehead rows) and smiling and/or squinting causes wrinkles at the corners of the eyes (lateral canthal lines or “crow's feet”). Static wrinkles result from a loss of elasticity in skin, which may arise from a variety of factors, including sun damage, poor nutrition, smoking, and genetic factors. Wrinkle folds, which may appear as deep grooves between the nose and mouth, arise from the sagging of underlying facial structure. Generally, the methods of this invention are suitable for treatment of all types of wrinkles. In certain embodiments of this invention, the wrinkles that are treated with topical chemodenervating agents are dynamic wrinkles.

The term “chemodenervating agent,” as used herein, refers to a substance that prevents a nerve from stimulating its target tissue, e.g. a muscle, a gland or another nerve. Generally speaking, chemodenervating agents act by interrupting nerve impulse transmission across a neuromuscular or neuroglandular junction, thereby blocking or reducing neuronal exocytosis of a neurotransmitter, or altering the action potential at a sodium channel voltage gate of a neuron. Non-limiting examples of chemodenervating agents contemplated by the invention include botulinum toxin, tetanus toxin, saxitoxin, and tetrodotoxin, and all serotypes and all combinations thereof.

The term “chemodenervation” encompasses all effects which directly or indirectly are induced by the chemodenervating agent, therefore also comprising upstream, downstream or long-term effects of said chemodenervating agent. Therefore presynaptic effects are also encompassed as well as postsynaptic effects, tissue effects and/or indirect effects via spinal or afferent neurons.

The term “botulinum toxin,” as used herein, refers to any of the known types of botulinum toxin, whether produced by the bacterium or by recombinant techniques, as well as any such types that may be subsequently discovered including engineered variants or fusion proteins. The botulinum toxin is selected from the group consisting of serotypes A, B, C, D, E, F, G and combinations thereof.

The term “low viscosity” as used herein, refers to a liquid having a viscosity in the range of approximately 0.0091 poise at 25° C., or 1 centipoise at 20° C. and substantially behaving in an aqueous manner.

In one aspect, the invention includes a stable low viscosity or liquid pharmaceutical formulation that includes, by way of example, isolated botulinum toxin and a buffer that is capable of providing a buffered pH range between about pH 5 and pH 6. According to this general embodiment, the toxin is mixed in a buffered liquid to form a low viscosity formulation which has a pH of between 5 and 6, particularly between about pH 5.4 and pH 5.8, and preferably about pH 5.5-5.6. The resulting formulation is stable for at least one year at room temperatures of between 10 and 30 degrees C. Generally, in accordance with the invention, any of the known botulinum toxin serotypes (e.g., serotypes A, B, C1, C2, D, E, F, or G) or other serotypes having equivalent biological activity, as well as other chemodenervating agents and toxins, may be incorporated into formulations of the invention. In preferred embodiments, the botulinum toxin used in the formulation is botulinum toxin serotype A isolated from Clostridium botulinum.

In preferred embodiments, botulinum toxin is present as a molecular weight complex in the formulation, at a concentration of about 100-20,000 U/ml, and particularly between about 1000-5000 U/ml. When Type A is used, it will generally be present at a concentration of about 20-2,000 U/ml, and particularly between about 100-1,000 U/ml. If combinations of different serotypes are used in the formulation, their useful dosage or concentration ranges can be determined in proportion to the dosages and concentrations exemplified herein, according to their respective biological activities. Buffers that can be used in the formulation are physiological buffers that are considered safe for injection into mammalian tissue, particularly into humans. Representative buffers include, but are not limited to phosphate, phosphate-citrate, succinate, acetate, citrate, aconitate, malate, and carbonate based buffer systems. Preferably, the formulation will also include an excipient protein, such as human serum albumin or gelatin, or an essential or semi-essential amino acid as is more fully set forth below.

Methionine, an essential amino acid, is one of the two sulfur-containing amino acids. The side chain is quite hydrophobic and methionine is usually found buried within proteins. Unlike cysteine, the sulfur of methionine is not highly nucleophilic, although it will react with some electrophilic centers. It is generally not a participant in the covalent chemistry that occurs in the active centers of enzymes.

Isoleucine, an essential amino acid, is one of the three amino acids having branched hydrocarbon side chains. It is usually interchangeable with leucine and occasionally with valine in proteins. The side chains of these amino acids are not reactive and therefore not involved in any covalent chemistry in enzyme active centers. However, these residues are critically important for ligand binding to proteins, and play central roles in protein stability.

Valine, an essential amino acid, is hydrophobic, and as expected, is usually found in the interior of proteins. Valine differs from threonine by replacement of the hydroxyl group with a methyl substituent. Valine is often referred to as one of the amino acids with hydrocarbon side chains, or as a branched chain amino acid.

Cysteine (abbreviated as Cys or C) is an α-amino acid with the chemical formula HO₂CCH(NH₂)CH₂SH. It is a semi-essential amino acid, which means that it can be biosynthesized in humans. The thiol side chain in cysteine often participates in enzymatic reactions, serving as a nucleophile. The thiol is susceptible to oxidization to give the disulfide derivative cystine, which serves an important structural role in many proteins. In a statistical analysis of the frequency with which amino acids appear in different chemical environments in the structures of proteins, free cysteine residues were found to associate with hydrophobic regions of proteins. Their hydrophobic tendency was equivalent to that of known non-polar amino acids such as methionine and tyrosine, and was much greater than that of known polar amino acids such as serine and threonine.

While free cysteine residues do occur in proteins, most are covalently bonded to other cysteine residues to form disulfide bonds. Disulfide bonds play an important role in the folding and stability of some proteins, usually proteins secreted to the extracellular medium Since most cellular compartments are reducing environments, disulfide bonds are generally unstable in the cytosol with some exceptions as noted below.

Disulfide bonds in proteins are formed by oxidation of the thiol groups of cysteine residues. The other sulfur-containing amino acid, methionine, cannot form disulfide bonds. More aggressive oxidants convert cysteine to the corresponding sulfinic acid and sulfonic acid. Cysteine residues play a valuable role by crosslinking proteins, which increases the rigidity of proteins and also functions to confer proteolytic resistance (since protein export is a costly process, minimizing its necessity is advantageous).

The essential and semi-essential amino acids may be advantageously employed to stabilize the toxin compound and permit it to be released over an extended period of time once it has been applied topically and has travelled transdermally.

It is appreciated that equivalents of the foregoing exemplary buffers, excipient proteins and essential and semi-essential amino acids will be recognized and utilized by persons having skill in the art. The toxin formulation of the invention may be packaged in any of a variety of containers or vials known in the art, while retaining its potency.

In a related aspect, the invention includes a method of treating a patient in need of inhibition of cholinergic transmission, such cholinergic transmission to selected muscle or muscle group or to a specific gland region, such as sweat glands (cutaneous disorder is hyperhydrosis), or to a particular organ having cholinergic innervation. In another embodiment, the present invention provides methods for treating cutaneous disorders comprising the step of administering any of the pharmaceutical formulations of the present invention to a subject in need thereof in an amount sufficient to reduce a sebaceous or mucous secretion.

In some cosmetic applications, the botulinum toxin formulations of the present invention may be administered to the muscles of the face, including the forehead and eye area, to reduce lines and wrinkles. The disclosed botulinum toxin formulations may be administered through a variety of modalities including surface application, subcutaneous and intramuscular injection. Specifically, botulinum toxin may be used, for example, to treat glabellar frown lines, crow's feet, horizontal forehead lines, nasolabial fold, mental crease, upper lip, platysmal bands, horizontal neck lines and wrinkles of the lower part of the face.

The present invention also encompasses a method of reducing neurotransmitter release in a subdermal structure of a patient, the method comprising the steps of non-chemically disrupting the stratum corneum of the patient's skin to reduce impermeability of the stratum corneum; and applying botulinum toxin to the skin of the patient in an area that has had the stratum corneum disrupted in the first step. The stratum corneum can be disrupted by abrasively removing the stratum corneum. Thus, the stratum corneum can be disrupted by applying a liquid gel to the patient's skin, and removing the adhesive material applied thereto.

Alternately, the stratum corneum can be disrupted by applying ultrasound at a frequency between 20 kHz and less than 10 MHz at an intensity that does not permanently damage the patient's skin. Or the stratum corneum can be disrupted by passing electrical current from a first point on the patient's skin to a second point on the patient's skin. The electrical current can be passed to create a plurality of pores in the stratum corneum to enhance passage of botulinum toxin to the subdermal structures. And the botulinum toxin can be applied in a pharmaceutical composition comprising an enhancing agent for enhancing the delivery of the botulinum toxin through the skin. Thus, the botulinum toxin can be is incorporated into a nano-micelles.

The present invention also encompasses a method of relieving pain in a patient caused by a spastic muscle, the method comprising the steps of (a) applying ultrasound at a frequency between about 10 kHz and 1 MHz to the patient's skin overlying the spastic muscle; and (b) applying botulinum toxin to the patient's skin that has received the ultrasound in step (a). Thus method can further comprise a step of abrasively removing portions of the stratum corneum of the patient's skin that received the ultrasound.

Examples of therapeutic and cosmetic treatments that can be treated using the botulinum toxin formulation but are not limited to other disease conditions such as hyperhidrosis, Acne etc. Preferably, the pharmaceutical formulations of the present invention are administered to the face or neck of the subject. In a preferred embodiment, the pharmaceutical formulations of the present invention are administered to the subject in an amount sufficient to reduce rhytides. Preferably, the formulation is administered between eyebrows of the subject in an amount sufficient to reduce vertical lines between the eyebrows and on a bridge of a nose.

The pharmaceutical formulations may also be administered near either one or both eyes of the subject in an amount sufficient to reduce lines at corners of the eyes. In another embodiment, the pharmaceutical formulations of the present invention may also be administered to a forehead of the subject in an amount sufficient to reduce horizontal lines on said forehead. In yet another embodiment of the present invention the pharmaceutical formulation is administered to the neck of the subject in an amount sufficient to reduce muscle bands in the neck.

It is the discovery of the present invention that botulinum toxin can be made and stored in a stable liquid formulation that retains its potency for an extended period of time, e.g., at least 1-2 years, at “refrigerator” temperatures (i.e., about 5±3° C., or more specifically, about 4±2° C., or more generally, 0-10° C.) or at least a “room temperature” (i.e., about 25° C., or more generally 10-30° C.). Such a formulation can be conveniently dispensed to humans or other mammalian species as a pharmaceutical without further re-constitution by the physician. The formulation is characterized by a pH of between about pH 5 and 6, preferably about pH 5.5-5.6, as maintained by appropriate buffering conditions. The formulation may also include one or more excipient proteins.

The diluent referred to above can be any pharmaceutically acceptable liquid which will not adversely affect the stability of the complex, and which supports a stable pH range between about pH 5 and pH 6. Examples of particularly suitable buffers include succinate and phosphate buffers; however, those of skill in the art will recognize that formulations of the invention will not be limited to a particular buffer, so long as the buffer provides an acceptable degree of pH stability, or “buffer capacity” in the range indicated.

Generally, a buffer has an adequate buffer capacity within about 1 pH unit of its pK. (Lachman, et al., 1986). In the context of the present invention, this includes buffers having pK's in the range of about 4.5-6.5. Buffer suitability can be estimated based on published pK tabulations or can be determined empirically by methods well known in the art. In addition to the succinate and phosphate buffers mentioned above, other pharmaceutically useful buffers include acetate, citrate, aconitate, malate, and carbonate (Lachman). The pH of the solution can be adjusted to the desired endpoint within the range using any pharmaceutically acceptable acid, for example hydrochloric acid or sulfuric acid, or base, for example sodium hydroxide. Succinate buffer was prepared in 3 L lots with 2.7 mg/mL disodium succinate and 5.8 mg/mL sodium chloride supplemented with 0.5 mg/mL

The term “excipient,” as used herein, refers to an inert material that can be used as a diluents or vehicle in the disclosed compositions, and which in some aspects and in certain amounts, may be suitable as hydrogel forming agents, as defined below. Suitable excipients include, for example, polyorthoester-compatible materials such as those listed in US Publication No. 2012/0041021. The term “excipient” may also include “excipient proteins.” Examples Of excipient proteins include, but are not limited to serum albumins, particularly human serum albumin, gelatin, chitosans, and the like. Such proteins will preferably be relatively non-immunogenic to the mammalian species into which the pharmaceutical formulation is to be administered. Excipients may also include dispersing agents or viscosity modulating agents.

These may include, without limitation, hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethyl-cellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 441,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents.

In a further preferred embodiment of the invention, small molecule peptides like, tri, tetra, penta, hexa, septa and octa peptides, Acetyl Hexapeptide-3 Cosmetic Topical Peptide, Melanotan II, ACVR2B (ACE-031), Argireline AcetateArgireline, Matrixyl Acetate(palmitoyl pentapeptide, peptide GHK spontaneously complexes with copper, Palmitoyl Tetrapeptide-3, and derivatives and analogues, (e.g., Argireline NP, Acetyl Glutamyl Heptapetide, Matrixyl, Snap-8, Syn-Tacks, Syn-Coll, Syn-Hycan, Leuphasyl, Pepha-Tight, Tego Pep 4-17 and Trylagen) are employed in combination with the toxin to enhance the stability and provide such stability during the topical application and transdermal passage of the toxin molecule. The peptides also enhance the long term, slow release of the toxin to permit the longer term chemodenervating effects thereof. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. As used herein, the term “hydrogel” means a matrix of crosslinked polymers capable of forming a solid substance. The hydrogel compositions described herein may be liquid at certain temperatures and solid at other temperatures, for example, a liquid at 4 degrees C. and a solid at 37 degrees C.

The term “hydrogel forming agent” means an agent that may be added to the compositions disclosed herein to form a hydrogel. Exemplary hydrogel forming agents include poloxamers, hyaluronan polymer, glycosaminoglycan polymer, keratan sulfate polymer (such as that disclosed in US Publication No. 2011/0171310), polysaccharides (e.g., HA, chitosan, chondroitin sulfate, alginate, carboxymethylcellulose), poly(ethyleneglycol), poly(lactic acid), poly(hydroxyethyl-methacrylate), poly(methylmethacrylate), proteins (e.g., elastin and collagen). Hydrogels of the present description can include more than one biocompatible polymer or hydrogel forming agent, such as, for example, 2, 3, 4, 5, 6, 7, 8, 9, 10, or more of such polymers or agents. The present composition according to the invention can moreover comprise at least one surfactant or a mixture of several surfactants.

By “surfactant” is meant within the meaning of the invention an emulsifying agent or a solubilizing agent. Within the framework of the invention the surfactants utilized can be chosen from the cationic, anionic or non-ionic surfactants.

Preferably the composition according to the invention comprises at least one surfactant chosen from the non-ionic surfactants of the group of polysorbates. Among the group of polysorbates, there can be mentioned polysorbate 20, polysorbate 21, polysorbate 40, polysorbate 60, polysorbate 61, polysorbate 65, polysorbate 80, polysorbate 81, polysorbate 85, polysorbate 120, polysorbate 80 acetate, Na lauryl sulfate, deoxycholate, chenodeoxycholate, polyoxyethylene, DMSO etc.

In another embodiment, an enhancing agent may be a vesicle that is able to store the neurotoxin within the vesicle. The vesicle can diffuse through the skin and thereby deliver the neurotoxin to the target site. The vesicle may be a lipid vesicle. In one specific embodiment, the neurotoxin is incorporated into a transfersome, which are deformable carries containing lipids and membrane softeners. The preferred surfactant according to a variant of the composition according to the invention is polysorbate 80.

A detailed embodiment of the invention is a pharmaceutical composition comprising a botulinum neurotoxin and a cross linked, polymeric, hyaluronic acid carrier for the botulinum neurotoxin, wherein the polymeric hyaluronic acid has a molecular weight between about 10,000 Daltons and about 20 million Daltons, the concentration of the polymeric hyaluronic acid in the formulation is between about 0.1 wt % and about 1 wt % and the viscosity of the pharmaceutical composition is between about 100 cps and about 1,000 cps at 25° C., at a shear rate of about 0.1/second. The botulinum neurotoxin is preferably a botulinum neurotoxin type A.

In one preferred embodiment, the carrier is a polymeric hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component preferably is in a range of about 50,000 Daltons or about 100,000 Daltons to about 1.3 million Daltons or about 2 million Daltons. In one embodiment, the present compositions include a polymeric hyaluronate component in an amount in a range about 0.05% to about 0.5% (w/v).

In a further useful embodiment, the hyaluronate component is present in an amount in a range of about 1% to about 4% (w/v) of the composition. In this latter case, the very high polymer viscosity forms a gel that slows particle sedimentation rate to the extent that often no resuspension processing is necessary over the estimated shelf life, for example, at least about 2 years, of the drug delivery system. Such a drug delivery system can be marketed in pre-filled syringes

In one embodiment of the invention, the enhancing agent is an alcohol. Examples of alcohols include short chain alcohols, such as alcohols having between about 2-5 carbon atoms. Some short chain alcohols include ethanol, isopropanol, methanol, and isobutanol, or combinations thereof. The alcohols may be mixed in the composition so that the concentration of alcohol in the composition is between about 10% and 40%. The alcohol may be admixed with glycerin to reduce potential irritation caused by higher concentrations of alcohol. Long chain alcohols are also useful to enhance the transdermal administration of neurotoxins, such as botulinum toxins. Examples of long-chain alcohols include alcohols having between about 8 and 12 carbon atoms, and some specific examples include n-dodekano, klenbuterol, and albuterol. Polyalcohols may also be used with the neurotoxin. Examples include propylene glycol, glycerol, polyethylene glycol, and dexpantheol, and combinations thereof.

The compositions of the invention may be used in an application device that permits application of the composition to a target site on the skin without applying the composition to non-target site areas of the skin. For example, a device may be employed that allows the composition to be applied without first applying the composition to one's fingers, which may lead to undesirable paralysis of the fingers. Suitable devices include spatulas, swabs, syringes without needles, and adhesive patches. Use of spatulas or swabs, or the like may require the device to be inserted into a container containing the composition. Using syringes or adhesive patches may be accomplished by filling the syringe or patch with the composition. The composition may then be topically spread by the spatulas or swabs, or may be expelled from the syringes onto the person's skin. Additional transdermal methods that non-chemically enhance the skin's permeability include low frequency ultrasound (20 kHz to 1 MHz).

Ultrasound is defined as sound at a frequency of between about 20 kHz and 10 MHz, with intensities of between 0 and 3 W/cm2. Low frequency ultrasound, as used herein, refers to ultrasound at a frequency that is less than 1 MHz, and preferably in the range of 20 kHz to 40 kHz. The ultrasound is delivered in pulses, for example, 100 msec pulses at a frequency of 1 Hz. The intensity of the ultrasound may vary between 0 and 1 W/cm2, and frequently varies between 12.5 mW/cm2 and 225 mW/cm2. Typical duration of exposure to ultrasound is between about 1 and about 10 minutes. The ultrasound is applied without causing an increase in skin temperature greater than about 1 degree Celsius. Low frequency ultrasound may be used alone or in combination with the composition to improve the permeability of the skin to the neurotoxin. Examples of ultrasound techniques for improving skin permeability may be found in U.S. Pat. Nos. 6,002,961 and 5,814,599. Surprisingly, it has been discovered that low frequency ultrasound, when applied in conjunction with a composition containing a botulinum toxin, permeabilizes the skin.

Additionally, the ultrasound may be delivered prior to application of the botulinum toxin to the skin. It has been discovered that low frequency ultrasound when applied before the topical application of botulinum toxin, temporarily disrupts the stratum corneum so that subsequent topical application of botulinum toxin achieves a therapeutic effect. In other words, the disruption caused by the ultrasound persists for several minutes, for example between about 10 and 30 minutes, to provide relatively easy transdermal delivery of botulinum toxin to the patient.

### EXAMPLES

The following examples illustrate aspects of our invention:

### Example 1

### Low Viscosity Botulinum Toxin-Hyaluronic Acid Formulation

A botulinum toxin-hyaluronic acid formulation can be prepared as follows. 1 gram of 1,4-butanediol diglycidyl ether (as cross linker) is added to a 1-L aqueous solution containing 10 g hyaluronic acid (as the viscous carrier), adjusted to pH 12 while vortexing. The molecular weight of the uncross linked hyaluronic acid is about 500,000 Daltons. The reaction mixture is incubated at 60° C. for 45 minutes and neutralized with glacial acetic acid. The resulting crosslinked hyaluronic acid can have a crosslinking density of about 10%. Ten milligrams of the crosslinked hyaluronic acid is added to 1 mL of an aqueous solution containing 9 mg sodium chloride, 5 mg human albumin USP and 1,000 mouse LD50 units of botulinum toxin type A complex. An aliquot of the lyophilized formulation containing 100 mouse LD50 units of toxin and 1 mg of the crosslinked hyaluronic acid is reconstituted with 1 mL of succinate buffer or with saline. Essential and semi-essential amino acids may also be substituted and multiplexed molecular penetration enhancers added to the combination. The resulting solution has a hyaluronic acid concentration of about 0.1 wt % and a viscosity of about 300 cps.

Palmitoyl Tetrapeptide-3 Octinoxate 7.5%
Acetyl Hexapeptide-8
Oxybenzone 4.0%
Avobenzone 2.0%
Cetearyl Alcohol 3.3%
Butylene Glycol 1.5%
C12-15 Alkyl Benzoate Cyclopentasiloxane 1.5%
Glycerin Ethoxydiglycol 1.0%
Na Hyaluronate 1.0%
Sodium Lauryl sulfate 0.75%
Alcohol 1.5%
Water/Eau qs to 1 mL

### Example 2

### Low Viscosity Botulinum Toxin-Hyaluronic Acid Formulation with a Higher Hyaluronic Acid Concentration

Another botulinum toxin-hyaluronic acid formulation can be prepared as follows. Twenty milligrams of the crosslinked hyaluronic acid is added to 1 mL of an aqueous solution containing 9 mg sodium chloride, 5 mg human albumin USP and 1,000 mouse LD50 units of botulinum toxin type A complex. An aliquot of the lyophilized formulation containing 100 mouse LD50 units of toxin and 1 mg the crosslinked hyaluronic acid is reconstituted with 1 mL of water for injection (WFI) or with saline for injection. The resulting solution has a hyaluronic acid concentration of about 0.5 wt % and a viscosity of about 300 cps. Since the amount of cross linking is decreased in the Example 2 formulation the concentration of the hyaluronic acid in the formulation is increased to provide the same viscosity as the Example 1 formulation. Essential and semi-essential amino acids may also be substituted and multiplexed molecular penetration enhancers added to the combination.

### Example 3

### High Viscosity Botulinum Toxin-Hyaluronic Acid Formulation

A high viscosity botulinum toxin-hyaluronic acid formulation can have the ingredients shown in Table 1 below.

Ingredient Amount
Botulinum toxin type A 100 units
Sodium hyaluronate (polymeric) 2.5% (w/v)
Palmitoyl Tetrapeptide-3 Octinoxate 7.5%
Acetyl Hexapeptide-8
Sodium chloride 0.63% (w/v)
dibasic sodium phosphate, 0.30% (w/v)
Monobasic sodium phosphate, 0.04% (w/v)
Succinate buffer q.s.
Sodium Lauryl sulfate 0.75%
Alcohol 1.5%
Viscosity at shear rate 170,000 ± 25% cps
0.1/second at 25° C.

Essential and semi-essential amino acids may also be substituted and multiplexed molecular penetration enhancers added to the combination.

### Example 4

A patient with brow furrows and botulinum toxin to reduce the wrinkles. The patient is asked to lay down. A suspension of BOTOX® and transfersomes as described above is topically applied to the patient's forehead. An ultrasound device massage over the forehead (frontalis) was applied to the formulation treated the patient's skin at a frequency of 15 kHz for a period of 10 minutes. The ultrasound energy is pulsed to reduce damaging the patient's skin. After 15 minutes, the ultrasound device massage was stopped was the suspension evaporated or absorbed into skin completely. The patient is instructed to wash his face approximately 6 hours later. In about 2-3 days, the patient begins to notice that the forehead wrinkles are reduced in number. Patients were followed for 3, 4 and 10 weeks for safety and efficacy using a visual analog scale for patient global self-assessment and a photo-scale rating severity of rhytides. Surprisingly, the composition described herein produced a substantial improvement within 72 hours of treatment with 75% of cases improved as assessed using a physicians grading scale and over 62% improvement using a patient self assessment scale (p. 0.01, compared to controls). To achieve similar results, at least 100-200 U of BOTOX® is necessary. The effects of the BOTOX® last for about 3-4 months.

Advantages of our formulations include increasing residency of the botulinum neurotoxin which will increase the efficiency of deactivating nerve terminals in a given muscle and potentially increase the duration of the muscle paralysis. Additionally, increasing the residency time of the botulinum neurotoxin in the muscle tissue can also reduce exposure of the botulinum neurotoxin to the lymphatic system.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A stabilized, low viscosity protein composition for topical application and transdermal delivery of an active agent for therapeutic use or cosmetic improvement in humans, said composition comprising a hydrogel forming combination of collagen, elastin, or a combination thereof, one or more absorption enhancers selected from a groups consisting of short chain alcohols, long chain alcohols, or polyalcohols, amines and amides, comprising urea, amino acids or their esters, amides, AZONE(R), derivatives of AZONE(R), pyrrolidones, or derivatives of pyrrolidones, terpenes and derivatives of terpenes, fatty acids and their esters, macrocyclic compounds, tensides, sulfoxides, liposomes, transfersomes, lecithin vesicles, ethosomes, water surfactants polyols, small molecule tri, tetra, penta, hexa, septa and octa peptides, Acetyl Hexapeptide-3 Cosmetic Topical Peptide, Melanotan II, ACVR2B (ACE-031), Argireline AcetateArgireline, Matrixyl Acetate (palmitoyl pentapeptide, peptide GHK spontaneously complexes with copper, Palmitoyl Tetrapeptide-3, and derivatives and analogues, (e.g., Argireline NP, Acetyl Glutamyl Heptapetide, Matrixyl, Snap-8, Syn-Tacks, Syn-Coll, Syn-Hycan, Leuphasyl, Pepha-Tight, Tego Pep 4-17 and Trylagen) and a pharmaceutically acceptable buffer capable of providing a buffered pH range to the composition of between about pH 5 and about pH 6, sodium chloride and at least one therapeutic or cosmetic concentration of an active agent encapsulated in a micelle formed by a combination of surfactants, solvents and stabilizers and wherein said protein composition is stable in low viscosity form at room temperatures of between 10 and 30 degrees C. for a period in excess of six months.

2. A composition according to claim 1 wherein the active agent is selected from the group consisting of a chemodenervating agent, hyaluronic acid, antioxidants, hormones, growth factors, vaccine agents, drugs, vasodilators, therapeutic proteins, small molecules, amines, peroxides, antiperspirant agents, analgesics, and combinations thereof.

3. A composition according to claim 1 wherein said hydrogel forming combination of materials comprises poloxamers, hyaluronan polymer, glycosaminoglycan polymer, sulfate polymer, polysaccharides, poly(ethyleneglycol), poly(lactic acid), poly(hydroxyethyl-methacrylate), poly(methylmethacrylate), proteins, or a combination thereof.

4. A composition according to claim 1 wherein said hydrogel forming combination of materials comprises a polysaccharide selected from hyaluronic acid, chitosan, chondroitin sulfate, alginate, carboxymethylcellulose, or a combination thereof.

5. A composition according to claim 1, wherein the chemodenervation agent is botulinum toxin.

6. A composition according to claim 1, wherein the active agent is hyaluronic acid.

7. A composition according to claim 5, wherein said botulinum toxin is type A and is present at a concentration of about 5,000±1000 U/ml in said composition.

8. A composition according to claim 5, further comprising hyaluronic acid.

9. A process for making a pharmaceutical composition, the process comprising the steps of preparing a pharmaceutical composition comprising a botulinum neurotoxin and a low viscosity carrier with skin absorption enhancers for the botulinum neurotoxin by mixing together the botulinum neurotoxin, the low viscosity carrier and small molecule tri, tetra, penta, hexa, septa and octa peptides, Acetyl Hexapeptide-3 Cosmetic Topical Peptide, Melanotan II, ACVR2B (ACE-031), Argireline AcetateArgireline, Matrixyl Acetate (palmitoyl pentapeptide, peptide GHK spontaneously complexes with copper, Palmitoyl Tetrapeptide-3, and derivatives and analogues, (e.g., Argireline NP, Acetyl Glutamyl Heptapetide, Matrixyl, Snap-8, Syn-Tacks, Syn-Coll, Syn-Hycan, Leuphasyl, Pepha-Tight, Tego Pep 4-17 and Trylagen) and a pharmaceutically acceptable buffer capable of providing a buffered pH range to the composition of between about pH 5 and about pH 6, sodium chloride.

10. A pharmaceutical composition comprising a botulinum neurotoxin type A and a cross linked, polymeric, hyaluronic acid carrier for the botulinum neurotoxin, wherein the polymeric hyaluronic acid has a molecular weight between about 10,000 Daltons and about 1 million Daltons, the concentration of the polymeric hyaluronic acid in the pharmaceutical composition is between 0.1 wt % and 0.5 wt % and the viscosity of the pharmaceutical composition is between 100 cps and about 500 cps at 25° C., at a shear rate of 0.1/second and small molecule tri, tetra, penta, hexa, septa and octa peptides, Acetyl Hexapeptide-3 Cosmetic Topical Peptide, Melanotan II, ACVR2B (ACE-031), Argireline AcetateArgireline, Matrixyl Acetate (palmitoyl pentapeptide, peptide GHK spontaneously complexes with copper, Palmitoyl Tetrapeptide-3, and derivatives and analogues, (e.g., Argireline NP, Acetyl Glutamyl Heptapetide, Matrixyl, Snap-8, Syn-Tacks, Syn-Coll, Syn-Hycan, Leuphasyl, Pepha-Tight, Tego Pep 4-17 and Trylagen) and a pharmaceutically acceptable buffer capable of providing a buffered pH range to the composition of between about pH 5 and about pH 6, sodium chloride.

11. A product by the process of claim 9.

12. A method of treating facial frown lines, facial wrinkles, wrinkles of the skin, wrinkles of the contour of the eye, glabellar frown lines, baldness, acne, excessive perspiration or hair loss comprising administering the composition of claim 1 in an amount effective to treat such condition.

13. A topical composition comprising (i) at least one active agent; (ii) a first compound, and (iii) a second compound, wherein the first compound and second compound are different, and each is selected from the group consisting of N-lauroyl sarcosine, sodium octyl sulfate, methyl laurate, isopropyl myristate, oleic acid, glyceryl oleate and sodium lauryl sulfoacetate and small molecule tri, tetra, penta, hexa, septa and octa peptides, Acetyl Hexapeptide-3 Cosmetic Topical Peptide, Melanotan II, ACVR2B (ACE-031), Argireline AcetateArgireline, Matrixyl Acetate (palmitoyl pentapeptide, peptide GHK spontaneously complexes with copper, Palmitoyl Tetrapeptide-3, and derivatives and analogues, (e.g., Argireline NP, Acetyl Glutamyl Heptapetide, Matrixyl, Snap-8, Syn-Tacks, Syn-Coll, Syn-Hycan, Leuphasyl, Pepha-Tight, Tego Pep 4-17 and Trylagen) and a pharmaceutically acceptable buffer capable of providing a buffered pH range to the composition of between about pH 5 and about pH 6, sodium chloride.

14. A method for removing wrinkles in a subject's forehead, the method comprising: producing a map of a forehead muscle of the subject; using the map, locating a target volume of the forehead muscle, wherein the target volume is between about 2 mm and about 12 mm below an epidermal surface of the subject; and delivering energy to the target volume at a power, a frequency, and for a time selected such that the energy creates a pattern of lesions in the target volume, the pattern selected to achieve a desired degree of paralysis of the muscle, wherein each of the lesions in the pattern is confined within the target volume and the delivered energy does not significantly damage tissue surrounding the target volume, and topically apply the composition of claim 1 in an amount effective to treat such condition.

15. The method of claim 14, wherein the delivered energy is selected to be ultrasound.

16. The method of claim 14, wherein the delivered energy is selected to be radio frequency electromagnetic energy.

17. The method of claim 16, wherein the selected frequency is within a range of 4 to 8 MHz; and the selected power is within a range of 60 to 80 W.

18. A method comprising the steps of: (a) non-chemically disrupting the stratum corneum of the patient's skin to reduce impermeability of the stratum corneum: (b) applying a fluid to the patient's skin; (c) applying a transdermal formulation to the skin of the patient in an area that had the stratum corneum disrupted in step (a), comprising; (i) a pharmaceutical composition comprising a stabilized botulinum toxin provided in a dried state and an enhancing agent that is mixable with the stabilized botulinum toxin provided in a dried state and facilitates transdermal administration of a botulinum toxin in a bioactive form to a subdermal target site of a human patient

19. The method of claim 18, wherein the stratum corneum is disrupted by applying ultrasound at a frequency between 20 Khz to 1 MHz at an intensity that does not permanently damage the patient's skin.

20. The method of claim 18, wherein, the stratum corneum is disrupted by passing an electrical current from a first point on the patient's skin to a second point on the patient's skin.
